(19) United States
(12) Patent Application Publication
(10) Pub. No.: US 2020/0037857 A1
(43) Pub. Date: Feb. 6, 2020

(54) SYSTEMS, METHODS AND DEVICES FOR PERFORMING WATER-AIDED ENDOSCOPY

(71) Applicant: Endeau, Inc., Escondido, CA (US)

(72) Inventors: Allen NEWMAN, Rancho Santa Fe, CA (US); Dave MATSUURA, Solana Beach, CA (US)

(21) Appl. No.: 16/341,406

(22) PCT Filed: Oct. 11, 2017
(86) PCT No.: PCT/US2017/056226

§ 371 Date: Apr. 11, 2019

Related U.S. Application Data
(60) Provisional application No. 62/406,840, filed on Oct. 11, 2016.

(51) Int. Cl. A61B 1/00

(52) U.S. Cl. CPC A61B 1/00119; A61B 1/00137

(57) ABSTRACT

Systems, methods and devices for performing water-aided endoscopy (WAE) are provided herein, including an endoscopic plug with a bulbous distal portion and a separate compressible exterior housing which are designed to create a sealed environment within the body cavity. The endoscopic plug may additionally include valves, balloons and pumps which provide means for introducing and removing liquid into the body cavity and maintaining the sealed environment to prevent fluid from within the body cavity from leaking to an external environment. The endoscopic plug provides a channel for insertion of specialized tools designed to interface with the multi-functional plug for insertion into the body cavity while maintaining the seal.

### BACKGROUND

### Field of the Invention

The embodiments described herein are related to systems, methods and devices for performing water-aided endoscopy (WAE), and more particularly to an endoscopic plug and compressible exterior housing which creates and maintains a sealed environment within a body cavity for performing WAE.

### Related Art

Endoscopy is a minimally invasive medical procedure where an endoscope is inserted into a body cavity in order to view the interior of the body cavity. If the body cavity has no orifice to the external environment, an opening must be created through the skin for insertion of the endoscope. If the body cavity has an existing orifice, such as the colon or esophagus, the endoscope can be inserted through the orifice without requiring a separate opening. Once the opening is created, a gas—either air or carbon dioxide—is inserted into the cavity to inflate the body cavity for better viewing by the endoscope. Although endoscopy may be used simply to view the body cavity for diagnostic purposes, one or more tools may be inserted into the body cavity at the same time in order to perform an interventional diagnostic or therapeutic procedure.

Water-aided endoscopy (WAE) is a type of endoscopy where the body cavity is filled with water instead of a gas. WAE provides several benefits over gas insufflation, including less distention of the body cavity and image magnification, but also provides significant challenges. While some leakage of air in an endoscopic procedure is expected and does not cause problems, leakage of water from WAE can cause many problems during the procedure, as leaking water may interfere with an individual performing the procedure and must be cleaned up. In addition, many of the tools used during a normal endoscopic procedure may not be suitable for use in a fluidic environment or perform the same way in WAE even if they are suited for exposure to liquid.

### SUMMARY

Embodiments described herein provide systems, methods and devices for performing water-aided endoscopy (WAE), including an endoscopic plug with a bulbous distal portion and a separate compressible exterior housing which are designed to create a sealed environment within the body cavity. The endoscopic plug may additionally include valves, balloons and pumps which provide means for introducing and removing liquid into the body cavity and maintaining the sealed environment to prevent fluid from within the body cavity from leaking to an external environment. The endoscopic plug provides a channel for insertion of specialized tools designed to interface with the multi-functional plug for insertion into the body cavity while maintaining the seal.

Other features and advantages of the present invention will become more readily apparent to those of ordinary skill in the art after reviewing the following detailed description and accompanying drawings.

### BRIEF DESCRIPTION OF THE DRAWINGS

The structure and operation of the present invention will be understood from a review of the following detailed description and the accompanying drawings in which like reference numerals refer to like parts and in which:

FIG. 1 is a perspective view illustration of a multi-functional plug and inserted plug introducer, according to an embodiment of the invention;

FIG. 2 is a perspective view of the multi-functional plug separated from the plug introducer, according to an embodiment of the invention;

FIG. 3 is a cross sectional view of the multi-functional plug, according to an embodiment of the invention;

FIG. 4A is a perspective view illustration of a compressible exterior housing for the multi-functional plug with a proximal base portion and a distal bulbous portion, and an inserted housing introducer, according to an embodiment of the invention;

FIG. 4B is a cross sectional view of the compressible exterior housing and inserted housing introducer protruding through a channel in the compressible exterior housing which includes a distal bulbous cavity, according to an embodiment of the invention;

FIG. 5A is a transparent view of the compressible exterior housing illustrating the channel and interior bulbous cavity along with the inserted housing introducer, according to an embodiment of the invention;

FIG. 5B is a transparent view of the compressible exterior housing separated from the housing introducer, according to one embodiment of the invention;

FIG. 6A is a perspective view of the multi-functional plug with inserted plug introducer separated from the compressible exterior housing, according to one embodiment of the invention;

FIG. 6B is a perspective view of the multi-functional plug and inserted plug introducer after insertion into the compressible exterior housing, according to one embodiment of the invention;

FIG. 7 is a perspective view of the plug introducer after being withdrawn from the multi-functional plug after insertion into the compressible exterior housing, according to one embodiment of the invention;

FIG. 8 is a cross sectional perspective view of the multi-functional plug inserted into the compressible exterior housing, according to an embodiment of the invention;

FIG. 9 is a transparent perspective view of the multi-functional plug inserted into the compressible exterior housing, according to an embodiment of the invention;

FIG. 10A is a cross sectional view of the multi-functional plug and plug introducer inserted into the compressible exterior housing, according to an embodiment of the invention;

FIG. 10B is a cross sectional view of the multi-functional plug inserted into the compressible exterior housing, according to an embodiment of the invention;

FIG. 11A is a perspective view illustration of a method of inserting the compressible exterior housing into a body orifice using the housing introducer, according to one embodiment of the invention;

FIG. 11B illustrates the step of inserting the compressible exterior housing and housing introducer inside the body orifice, according to one embodiment of the invention;

FIG. 11C illustrates the step of removing the housing introducer from the compressible exterior housing after the compressible exterior housing is inserted into the body orifice, according to an embodiment of the invention;

FIG. 11D illustrates the step of inserting the multi-functional plug into the channel of the compressible exterior housing using the plug introducer, according to an embodiment of the invention;

FIG. 11E illustrates the step of removing the plug introducer from the multi-functional plug after the multi-functional plug has been inserted into the compressible exterior housing, according to an embodiment of the invention;

FIG. 11F illustrates the multi-functional plug and compressible exterior housing positioned within the body orifice as a result of the method, according to an embodiment of the invention; and

FIG. 12 is a flow diagram illustrating an example process for inserting a multi-functional plug into a body orifice, according to an embodiment of the invention.

### DETAILED DESCRIPTION

Certain embodiments disclosed herein provide for devices used for performing water-aided endoscopy (WAE), including an endoscopic plug with a bulbous distal portion and a separate compressible exterior housing which are designed to create a sealed environment within the body cavity. The endoscopic plug may additionally include valves, balloons and pumps which provide means for introducing and removing liquid into the body cavity and maintaining the sealed environment to prevent fluid from within the body cavity from leaking to an external environment. The endoscopic plug provides a channel for insertion of specialized tools designed to interface with the multi-functional plug for insertion into the body cavity while maintaining the seal. The endoscopic plug is therefore configured to ease the process of insertion of endoscopic tools during an endoscopic procedure, as well as to increase the retention of the endoscopic plug in a fixed position to prevent leakage or discomfort to a patient during the endoscopic procedure.

After reading this description it will become apparent to one skilled in the art how to implement the invention in various alternative embodiments and alternative applications. However, although various embodiments of the present invention will be described herein, it is understood that these embodiments are presented by way of example only, and not limitation. As such, this detailed description of various alternative embodiments should not be construed to limit the scope or breadth of the present invention as set forth in the appended claims.

Endoscopic Plug

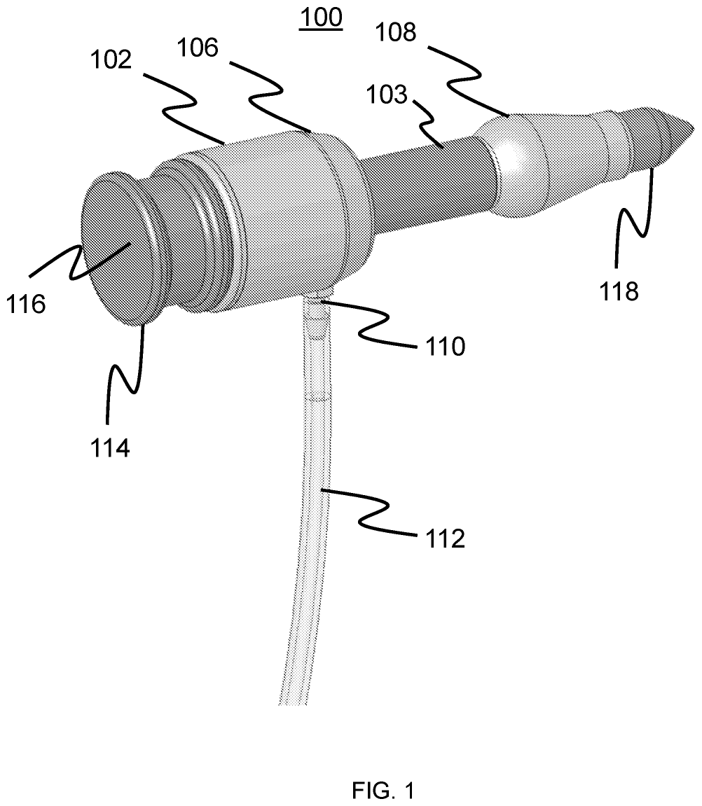

In one embodiment illustrated in FIG. 1, an endoscopic plug 100 is a tubular-shaped device with a proximal end 102 and a distal end 104 connected via a central shaft 103, where the distal end 104 is configured to be inserted into a body cavity, while the proximal end 102 is positioned just outside of a body cavity opening. Furthermore, a base pad 106 is positioned at the proximal end 102 for sealing and anchoring the plug 100 at the opening of the body cavity, while a bulbous portion 108 is formed at the distal end 104 for creating a seal with a tissue wall of the body cavity. The bulbous portion 108 has a diameter large enough to abut against the tissue wall of a body cavity opening (such as an intestinal wall) such that the bulbous portion 108 retains the plug 100 within the cavity and prevents the plug 100 from moving in either direction—further into the body cavity or out of the body cavity. In one embodiment, the bulbous portion 108 has a sloped angle on the distal and proximal portion, with the angle on the distal slope of the bulbous portion being less than the angle on the proximal slope in order to make insertion of the plug 100 easier. The endoscopic plug 100 is therefore designed to create two different seals—one inside the body cavity where the bulbous portion 108 abuts against tissue of the body cavity, and one at the opening of the body cavity where the base pad 106 abuts the body cavity opening. These two seals—one within the cavity and one at the opening—prevent fluid from leaking to an exterior environment, and also provide a more comfortable plug for the patient. In addition, the shape of the base pad 106 prevents rotation of the plug 100 within the body cavity.

During WAE, the fluid pressure within the body cavity should be maintained at a constant level. This may be accomplished through the use of a fluid tube 112 and fluid tube adapter 110, as shown in the embodiment in FIG. 1. The fluid tube 112 introduces fluid into the body cavity via the fluid tube adapter 110, which opens into a central opening 120 in the plug 100. The fluid tube adapter or fluid tube itself may also include a pressure release valve to help relieve fluid pressure within the body cavity if it reaches a certain threshold level. A separate fluid return tube (not shown) may be provided which allows fluid to exit the plug 100 in order to help maintain the constant fluid pressure level within the body cavity.

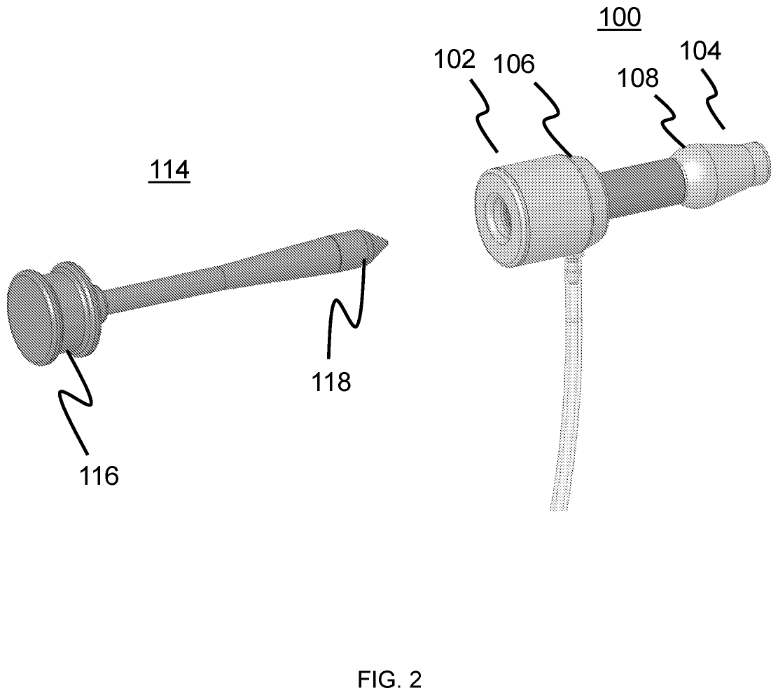

FIG. 1 also illustrates a plug introducer 114, which has a base handle 116 adjacent the base pad 106 and a narrow diameter tip 118 which fits into the plug 100. The base handle 116 of the introducer is used to manually insert the plug into the body cavity. One embodiment of the plug introducer 114 is more clearly illustrated in FIG. 2, which illustrates the plug introducer 114 separated from the plug 100 prior to being inserted into the plug 100.

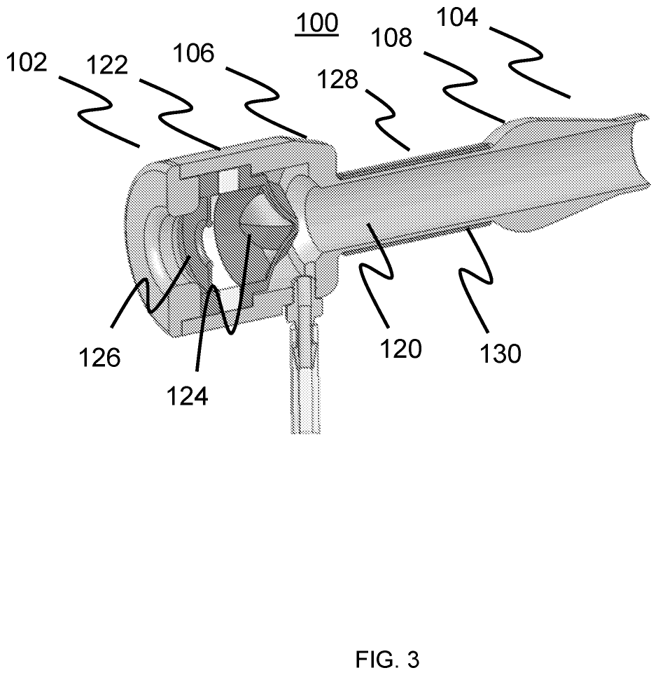

FIG. 3 is a cross sectional view of the endoscopic plug 100, further illustrating an central opening 120 which spans the length of the plug from the proximal end 102 to the distal end 104. The central opening 120 allows one or more endoscopic tools (not illustrated) to be introduced into the body cavity for use in an endoscopic procedure. Within the proximal end 102 and proximal to the base pad 106 is a base portion 122 which has a larger diameter than the middle and distal portions of the central opening 120. The base portion 122 may contain one or more valves and seals that prevent fluid from leaking through to the external environment. The type and number of valves or seals used in the base portion may vary, but in the illustrated embodiment, a distal valve 124 is an intermittent continuous flow valve, while a proximal seal 126 is provided as a secondary seal to prevent fluid from passing to the exterior environment. As can be seen from the illustration in FIG. 3, the intermittent continuous flow valve 124 maintains a seal to prevent liquid from escaping the plug regardless of whether an endoscopic instrument, introducer or other object has penetrated the central opening 120. In contrast, the proximal seal 126 is then designed with a permanent central opening which therefore maintains a seal when an endoscopic tool has been inserted into the body cavity. However, it is feasible to provide only a single valve/seal in the base portion that will prevent fluid from leaking regardless of whether an endoscopic tool has been inserted into the central opening or not. In one embodiment, the base portion 122 including the one or more valves and seals may be molded as a single component device in order to simplify the manufacturing of the plug.

In this embodiment in FIG. 3, the bulbous portion 108 does not change the diameter of the central opening 120 in the plug, such that the diameter of the central opening is approximately the same from the base pad 106 to the distal end 104. However, an alternative would be a hollowed-out bulbous portion 108 which may provide more compression and create a more comfortable exterior distal seal around the body cavity wall. Furthermore, in one embodiment the bulbous portion 108 may be a balloon which can be inflated and deflated to differing sizes and diameters in order to create a customized seal which for each patient. An air tube (not shown) may extend along a middle tubular portion 128 and in contact with the bulbous portion 108 in order to inflate or deflate the bulbous portion. The air tube may further extend to the exterior environment and be provided with a syringe, compressor or positive air pressure device which can maintain a needed level of air pressure to keep the bulbous portion inflated to a desired diameter. In a further embodiment, a second balloon may be provided to inflate a portion of the plug in a shape which prevents rotation, in addition to the bulbous portion 108 and base pad 106.

In a further embodiment, the middle tubular portion 128 located between the base pad 106 and bulbous portion 108 may be provided with a coating or sleeve 130 which reduces the friction on the body cavity wall as it is being inserted and removed. In one embodiment, the coating or sleeve 130 may be a silicone or other lubricious material.

While the shape of the base pad 106 provides a degree of rotation prevention in its current form, in an alternate embodiment, the base pad may be configured in a tear-drop shape to better conform to the anatomical features of human anatomy—such as the intergluteal cleft around the anus—in order to further prevent rotation of the endoscopic plug once it has been inserted. Additional details regarding the shape of the base pad is provided below with regard to the compressible exterior housing.

Compressible Exterior Housing

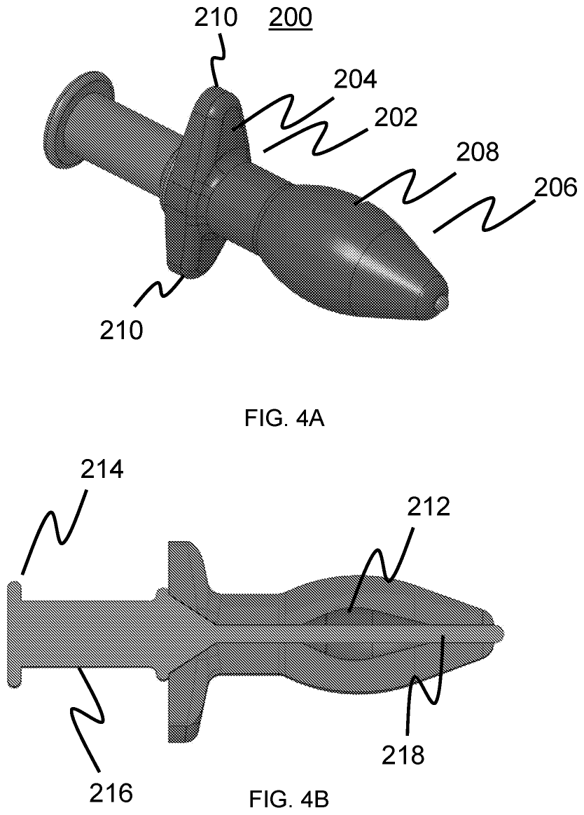

In one embodiment illustrated in FIG. 4A and FIG. 4B, a compressible exterior housing 200 may be inserted into the body cavity prior to insertion of the endoscopic plug 100 in order to more easily insert the plug and reduce the amount of discomfort for a patient. The compressible exterior housing 200 has a central opening extending along the length of the housing for insertion of the endoscopic plug 100, and is therefore designed to fit over the middle tubular portion 128 and distal portion 104 of the endoscopic plug, as will be illustrated and described further below. The compressible exterior housing 200 may be advantageous for patients with weak or scarred tissue and compromised musculature at the body cavity opening or immediately within the body cavity along tissue walls which may have been weakened by prior medical procedures, age or disease. Furthermore, the housing may be advantageous for pediatric colonoscopy.

The compressible exterior housing 200 is shaped similarly to the endoscopic plug 100 in that it includes a proximal end 202 with a base pad 204 designed to fit a body cavity opening, and a distal end 206 with a bulbous portion 208 designed to be inserted into the body cavity. The base pad is configured with two wing portions 210 which extend in opposite directions and which are shaped to fit within the intergluteal cleft. The wing portions 210 prevent the compressible exterior housing 200 from rotating while inserted into the body cavity. The bulbous portion 208 also has proximal and distal sloped portions which act to retain the housing 200 within the body cavity without allowing it to move further in or back out of the body cavity. Additionally, the bulbous portion 208 has side walls which are the same or similar thickness to the rest of the housing and which as a result provides a cavity 212 inside of the bulbous portion, as shown in FIG. 4B. This cavity allows the bulbous portion to compress more easily than the bulbous portion 108 of the endoscopic plug 100, providing less discomfort when it is inserted into the body cavity.

The base pad 204 and bulbous portion 208 are designed to provide a soft seal to prevent fluid from exiting the body cavity during WAE, and also to provide retention in order to prevent movement of the plug 100 once it is positioned in the body cavity. The compressible exterior housing 200 may be made from silicone or another compressible polymer which provides a sufficient amount of compression to avoid excessive expansion of the body cavity opening or excessive friction along the tissue walls of the body cavity as it is being inserted.

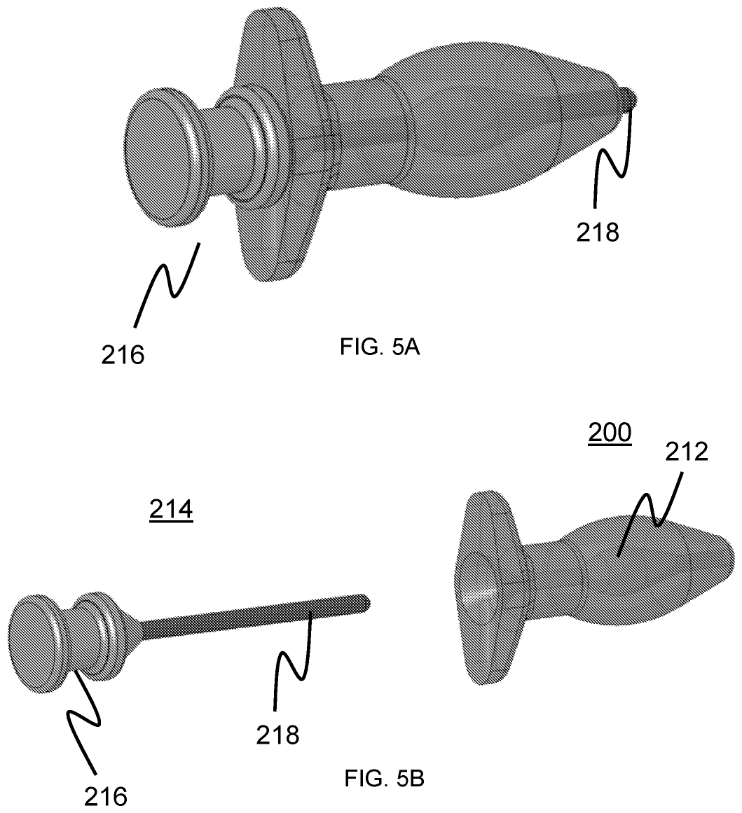

The compressible exterior housing 200 is shown in FIGS. 4A and 4B with its own housing introducer 214 that is utilized to insert the compressible exterior housing into the body cavity similarly to the plug introducer 114 and the endoscopic plug 100. The housing introducer 214 includes a base handle 216 and a narrow diameter tip 218 similar to the plug introducer 114. The housing introducer 214 is illustrated inside of the compressible exterior housing 200 in FIGS. 4A, 4B and FIG. 5A, but is shown separately in FIG. 5B. The narrow diameter tip 218 is substantially smaller in diameter than the plug introducer 114, which again reflects the smaller size of the compressible exterior housing and the additional ease in which it can be inserted into the body cavity without significant discomfort to the patient.

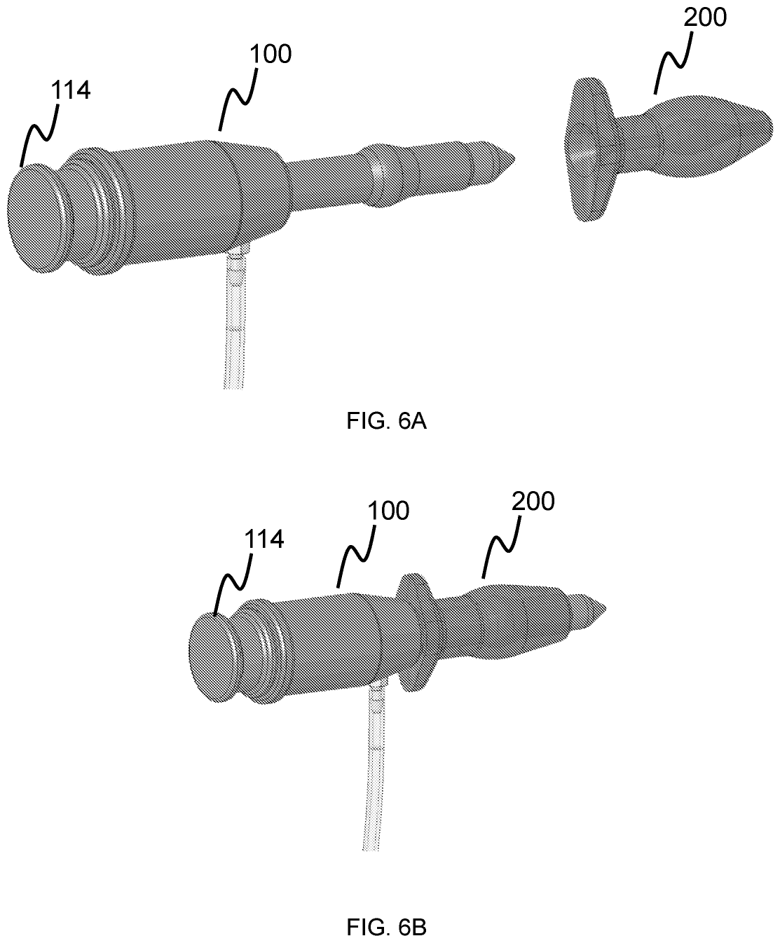

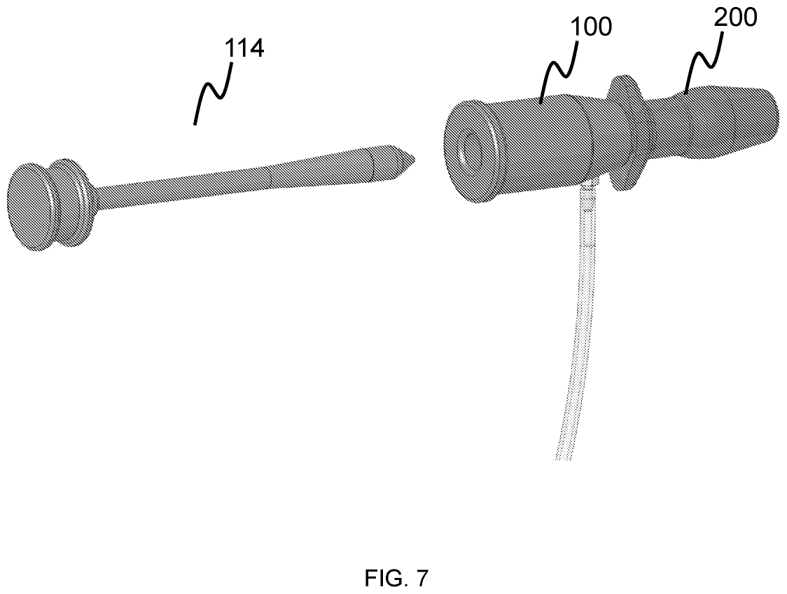

FIG. 6A is a perspective view of the endoscopic plug 100 with inserted plug introducer 114 prior to insertion into the compressible exterior housing 200, according to one embodiment of the invention, while FIG. 6B illustrates the endoscopic plug 100 and inserted plug introducer 114 after insertion into the compressible exterior housing 200. In FIG. 7, the plug introducer 114 is withdrawn from the plug 100 which is now fitted within the compressible exterior housing 200.

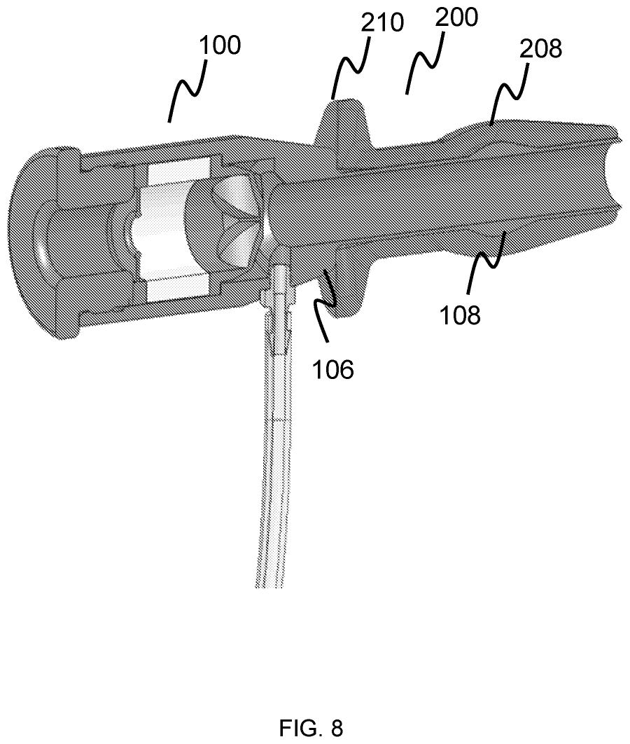

FIG. 8 is a cross sectional perspective view of one embodiment of the endoscopic plug 100 inserted into the compressible exterior housing 200, which more clearly illustrates how the bulbous portion 208 of the housing 200 is designed to fit over the bulbous portion 108 of the plug 100, creating a unique fit between the two pieces which is highly unlikely to move once position in the body cavity. FIG. 8 also illustrates how the wing portions 210 of the compressible exterior housing 200 abut the base pad 106 of the endoscopic plug 106, such that the base pad 106 helps to further retain the compressible exterior housing 200 within the body cavity.

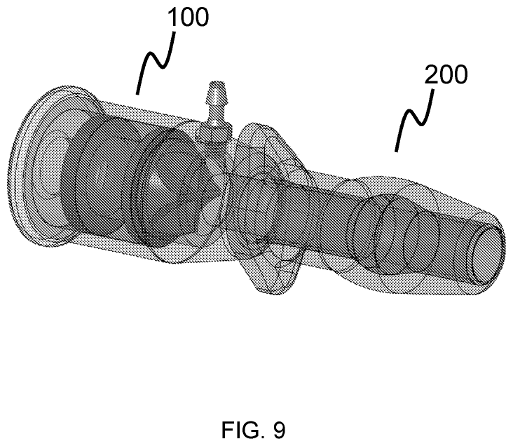

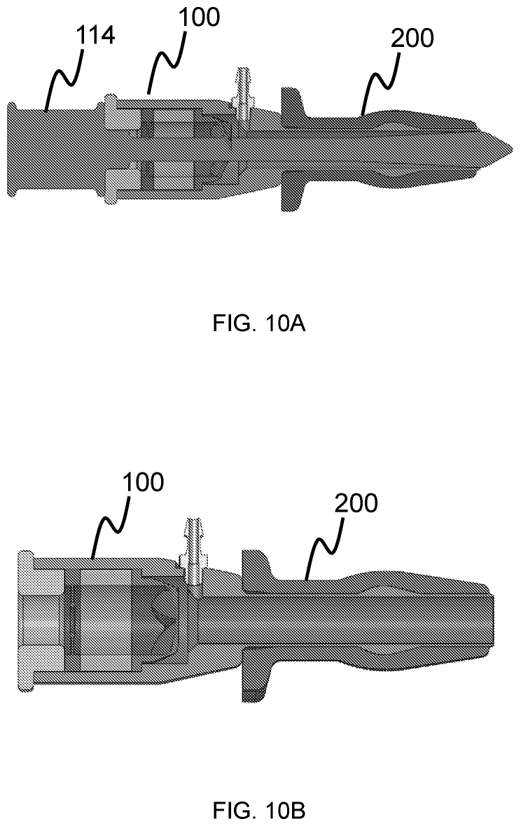

FIG. 9 is a transparent perspective view of the multi-functional plug 100 inserted into the compressible exterior housing 200, according to an embodiment of the invention, while FIG. 10A and FIG. 10B illustrate cross sectional views of the multi-functional plug 100 and plug introducer 114 inserted into and removed from within the plug 100, leaving the compressible exterior housing 200 and plug 100 in their inserted position within the body cavity.

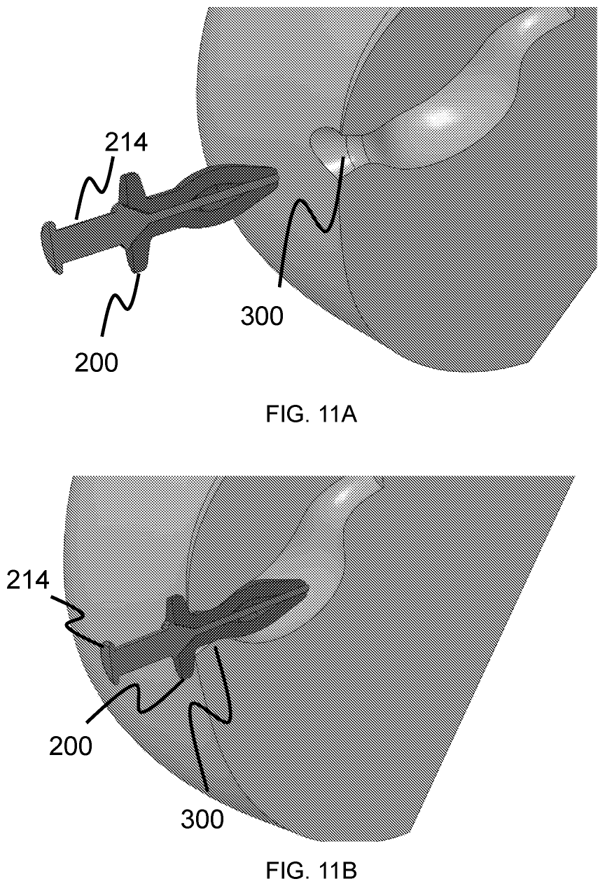

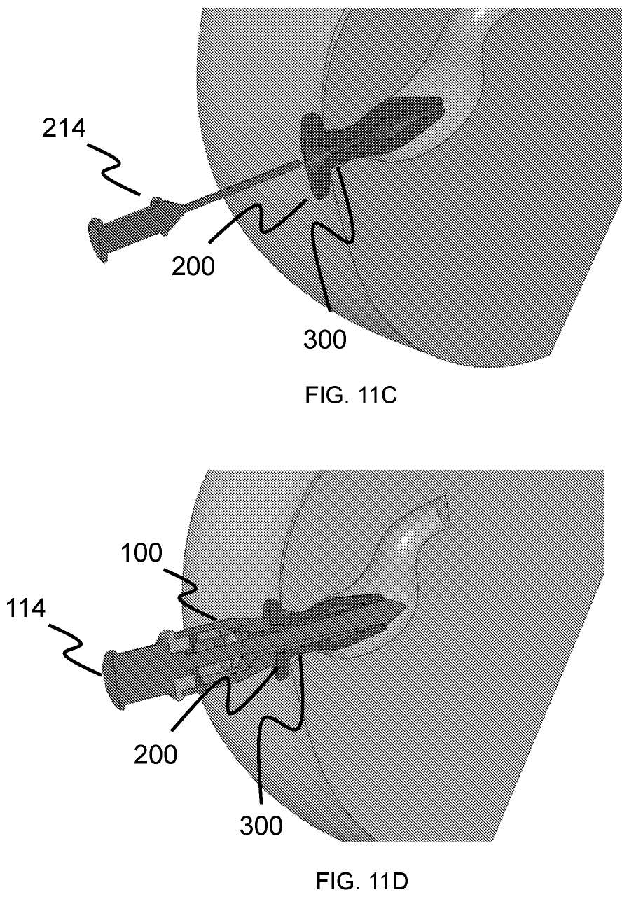

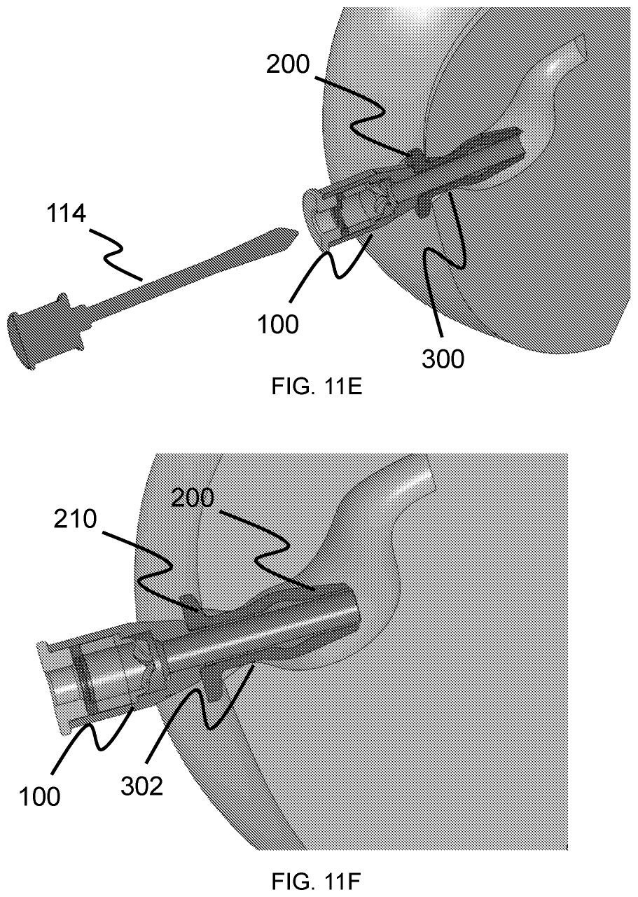

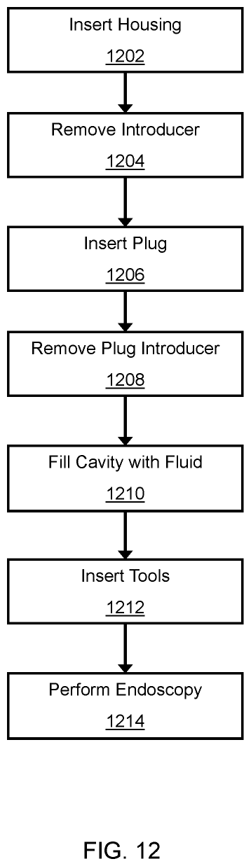

FIGS. 11A-11F illustrate one embodiment of a method shown in the flow diagram of FIG. 12 of inserting the compressible exterior housing 200 and endoscopic plug 100 into a body orifice using the housing introducer. In FIG. 11A and FIG. 11B (step 1202), the compressible exterior housing 200 is inserted into a body cavity 300 using the housing introducer 214, after which (step 1204), the housing introducer 214 is removed, as shown in FIG. 11C. In FIG. 11D (step 1206), the endoscopic plug 100 is inserted into the body cavity 300 and more specifically into the compressible exterior housing 200 using the plug introducer 114. The plug introducer 114 is then removed in FIG. 11E (step 1208), after which a patient is ready for a doctor or other medical professional to perform WAE. FIG. 11F illustrates how the wings 210 of the compressible exterior housing 200 fit against the body cavity opening to ensure that not only is the seal created, but rotation is prevented as well. Furthermore, the placement of the bulbous portion 108 in the plug 100 and the bulbous portion 208 on the housing 200 is also advantageous given the position of the anal sphincter 302, as the bulbous portions rest just inside of the sphincter muscles and allow the sphincter muscle 302 to contract over the bulbous portions and onto the middle tubular portion, further increasing the exterior seal around the plug and preventing movement of the plug 100 and housing 200.

Although not illustrated herein, once the plug and housing are inserted into the body cavity, the steps of WAE can be performed, as illustrated in the remaining steps of FIG. 12. The body cavity is filled with fluid (step 1210), after which one or more endoscopic tools may be inserted into the plug 100 (step 1212) to perform an endoscopic procedure (step 1214).

The above description of the disclosed embodiments is provided to enable any person skilled in the art to make or use the invention. Various modifications to these embodiments will be readily apparent to those skilled in the art, and the generic principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly not limited.

What is claimed is:

1. An endoscopic plug for water-aided endoscopy, comprising:
a base pad disposed at a proximal end of the endoscopic plug, wherein the base pad is shaped to create a water-tight seal between a body cavity and an external environment upon insertion into a body cavity opening;
a bulbous portion disposed at a distal end of the endoscopic plug, wherein the bulbous portion is shaped to create a water-tight seal with a tissue wall within a body cavity;
a shaft portion connecting the base pad with the bulbous portion; and
an opening extending axially from the proximal end of the endoscopic plug to the distal end of the endoscopic plug and configured to receive one or more endoscopic tools.

2. The endoscopic plug of claim 1, wherein the bulbous portion has a sloped angle on a proximal side extending to the shaft portion, and a sloped angle on a distal side extending to the distal end.

3. The endoscopic plug of claim 2, wherein the sloped angle on the distal side of the bulbous portion is less than the sloped angle on the proximal side of the bulbous portion.

4. The endoscopic plug of claim 1, further comprising at least one fluid connector for introducing fluid into the body cavity through the endoscopic plug.

5. The endoscopic plug of claim 1, further comprising at least one valve disposed within the opening within the base pad which seals the opening to prevent fluid within the body cavity from escaping to the external environment.

6. The endoscopic plug of claim 5, wherein the at least one valve is an intermittent continuous flow valve.

7. The endoscopic plug of claim 1, wherein an exterior of the shaft portion is covered by a frictionless sleeve.

8. The endoscopic plug of claim 1, further comprising a compressible exterior housing shaped to cover the shaft portion and the bulbous portion.

9. The endoscopic plug of claim 8, wherein the compressible exterior housing includes a central opening extending axially from a proximal end to a distal end in order to receive the endoscopic plug.

10. The endoscopic plug of claim 9, wherein the compressible exterior housing includes a base pad at the proximal end, the base pad including two wing portions extending from the central opening in opposing directions and shaped to fit an intergluteal cleft.

11. The endoscopic plug of claim 10, wherein the compressible exterior housing includes a housing bulbous portion at the distal end, wherein the housing bulbous portion is shaped to create a water-tight seal with a tissue wall within a body cavity.

12. The endoscopic plug of claim 11, wherein the compressible exterior housing includes a cavity disposed within the central opening and the housing bulbous portion to receive the bulbous portion of the endoscopic plug.

13. The endoscopic plug of claim 12, wherein the compressible exterior housing is made of silicone.

14. A method of inserting an endoscopic plug for water-aided endoscopy, comprising:
inserting a compressible exterior housing into a body cavity opening using a housing introducer;
removing the housing introducer to leave the compressible exterior housing inserted in the body cavity opening;
inserting an endoscopic plug into an opening in the compressible exterior housing using a plug introducer;
removing the plug introducer to leave the endoscopic plug inserted into the opening in the compressible exterior housing.
